(19) United States
(12) Patent Application Publication
(10) Pub. No.: US 2026/0036562 A1
(43) Pub. Date: Feb. 5, 2026

(54) METHOD FOR MEASURING AND EVALUATING MICROBIOLOGICAL PARAMETERS AND MAINTAINING FOOD SAFETY IN THE PRODUCTION PROCESS OF FOOD DERIVED FROM ANIMAL PROTEIN

(71) Applicant: Premex, Inc., Durham, NC (US)

(72) Inventors: Sebastián Vélez PÉREZ, Medellín (CO); Esteban Valencia YEPES, Medellín (CO); Melissa Ortega ALZATE, Medellín (CO); Verónica Yepes MEDINA, Medellín (CO); Juan Camilo Arias ROBLEDO, Wageningen (NL); Camilo MEJIA, Medellín, CO (US)

(21) Appl. No.: 18/792,691

(22) Filed: Aug. 2, 2024

(51) Int. Cl. G01N 33/02

(52) U.S. Cl. CPC G01N 33/02

(57) ABSTRACT

The present disclosure relates to a system and a method for the measurement, implementation, and maintenance of the safety index for microbiological analysis for food safety for producers of food derived from animal protein, in order to analyze the microbiological behaviors and the actual or potential risk levels of microorganisms (bacteria and fungi) in each of the necessary productive stages. The system and method allow for optimization, control, and prevention through the real-time determination of the safety index in production processes for the production in-situ of food derived from animal protein.

### TECHNICAL FIELD

The present disclosure relates to the technical field of processes measuring, implementing, and maintaining the safety index for the microbiological analysis of food safety for producers of food derived from animal protein. The present disclosure also relates to the technical field of the food industry, focused on the production of animal protein and, more particularly, to a computer-implemented disclosure that combines software, hardware, and a method to solve the technical problem raised in this application.

### BACKGROUND

There are multiple documents in the prior art where the analysis of food safety index in biological processes is performed during the measurement and processing of the safety index for microbiological analysis in the control of production processes in food production in general.

For example, patent application US20140309968 entitled “Systems and methods for processing food assessment data”, relates to a computer-implemented method for processing food assessment data. The method includes an interface to enable input of food assessment data and a database to store the assessment data. The method includes a processor to analyze the assessment data to produce a food assessment model indicating the safety and/or quality thereof.

Patent application WO 2021146624 entitled “Food safety performance management models”, relates to systems and/or methods that monitor and/or evaluate food safety performance by analyzing data from one or more data sources to monitor and/or evaluate food safety performance for a food establishment.

Patent application WO 2013071499 entitled “Method and apparatus for providing food safety identification and warning”, relates to a method for food safety identification and warning. The method involves processing and/or facilitating a processing of news information to cause, at least in part, an extraction of safety information related to one or more food items. The safety information includes, at least in part, one or more potential food safety incidents, one or more respective times of the one or more potential food safety incidents, one or more respective locations of the one or more potential food safety incidents, or a combination thereof. The approach also includes determining a number of the one or more potential food safety incidents with respect to the one or more food items, the one or more times, the one or more locations, or a combination thereof. The approach also includes determining one or more confirmed food safety incidents based, at least in part, on comparison of the number against one or more threshold criteria.

Patent application US20130089838 entitled “Food safety and risk analyzer”, relates to systems and methods for gathering, analyzing, querying, and providing food safety and risk profile information to end-users via a central or distributed database across a variety of communications modes.

Patent U.S. Pat. No. 9,477,962 entitled “Monitoring food products”, relates to various systems, methods, and computer program products to improve food safety in a food and beverage supply chain. The embodiments disclosed herein utilize prepared product tags to continuously monitor food and beverage temperatures. The temperatures and other associated data may be wirelessly transmitted to centralized locations for aggregation and storage. Mobile devices may directly communicate with the product tags to receive the temperature data. A host of management applications may utilize the data in order to ensure that safety rules and guidelines are met, while also providing valuable business insight.

Patent application EP 3539886 entitled “Method and system for tracking food safety data using hash trees”, relates to a method for tracking food data transactions in a network data processing system comprising the steps of reading a network storage devices, a packaged food production data hash tree, receiving a food data transaction from a hardware data processor in a food packaging machine, appending the food data transaction to the packaged food production data hash tree, and causing decentralized storage of identical instances of the appended packaged food production data hash tree in the plurality of network storage devices. This reference further provides a network data processing system and a computer program for carrying out the method.

Patent application WO 2017039473 entitled “System and procedure for managing the process of verification of the safety of food and agricultural products”, relates to a system and method of management and control of process and protocol for food and agricultural product safety inspection using information communication technologies and advance sensor and actuator systems.

Patent U.S. Pat. No. 9,811,788 entitled “Food safety management system”, relates to a food safety management system, including a web portal for management and reporting and a handheld computing device for checklist completion. A checklist of tasks to be performed in a food service establishment is obtained and displayed on a touchscreen of the handheld computing device. Confirmation is obtained on the touchscreen whether a task has been completed. An identifier at a location in the food service establishment is inputted to verify that a task has been completed. Temperature and humidity readings are obtained from one or more stationary sensors monitoring a food storage environment. Task completion data, temperature data, and humidity data are sent to a server.

Patent U.S. Pat. No. 7,412,461 entitled “System and method for identifying a food event, tracking the food product, and assessing risks and costs associated with intervention”, relates to a food safety system and method, providing a comprehensive consumer risk distribution model, which can be applied to any food item.

Patent application WO 2021176405 entitled “Health and safety management system”, relates to health and safety management system for use in worksite or hospitality premises, such as a restaurant. The system (100) comprises at least one source of health and safety related information of the worksite or hospitality premise and at least one at least one machine learning engine (1115). The machine learning engine (1115) is configured to receive the health and safety related information as at least one input data (1103). The machine learning engine (1115) is further configured analyze the at least one input data (1103) and predict at least one issue relating to health and safety at the worksite or the hospitality premises that requires attention by one or more authorized parties.

Patent application CN 112052467 entitled “Food safety big data sharing method” relates to a food safety big data sharing method. The method comprises the steps of multi-source information system integration, data encryption and desensitization, data standardization, data extraction and collection, data arrangement, coding analysis and information restoration, and hierarchical sharing.

According to the above, it is observed that, despite the diversity of systems, devices, methods, and/or procedures for conducting the food safety risk analysis based on data, or the management of microbiological data from different sources to establish a level of safety risk for food control in general, it is now necessary to provide, for the animal feed industry, processing plants, and primary production within the production process of food derived from animal protein, a system and a method for measuring, implementing, and maintaining the safety index in microbiological analysis of food safety for producers of food derived from animal protein which comprises more effective technical steps or stages, that are simpler and more agile when performing the analysis of safety index in the raw material, in the processes, and in the finished product that is carried out within an animal feed production plant, a processing plant, and in primary production, in order to improve the use of resources in all production processes in the production plant for the development of animal feed in an effective way and that in turn is a tool for optimization, control, and prevention through the determination in real time of the safety index in the production processes for the production in-situ of food derived from animal protein.

The documents of the referenced state of the art, disclose part of the state of the art of the same technological field as the application, as it is observed in patents US20140309968, WO2021146624, WO2013071499, US20130089838, U.S. Pat. No. 9,477,962, EP3539886, WO2017039473, U.S. Pat. Nos. 9,811,788, 7,412,461, WO2021176405, and CN112052467.

Based on the above, these documents, that are part of the state of the art, are referenced herein to explain the background of the present application and to take as a starting point the improvements that make the application different from what already exists through its technical characteristics, comprising its system and method, that solve in a different way the technical problem raised in this application.

### SUMMARY OF THE DISCLOSURE

In order to overcome the deficiencies of the prior art, the present disclosure provides a method for measuring and evaluating microbiological parameters and maintaining food safety in a production process for food derived from animal protein, comprising the steps of:
a. receiving a file with tabulated information on the microbiological results of one or more stages of the production process;
b. verifying whether the information received complies with preset variables wherein, if the information does not comply with the preset variables, it is corrected by means of a correction algorithm;
c. transforming the information by means of a transformation algorithm;
d. storing the information received in a data storage unit;
e. calculating food safety indexes of the production process and/or its stages from the information received using a calculation algorithm;
f. generating a report with the food safety indexes to determine an action in the production process and/or its stages to maintain food safety.

In one embodiment, the file is received manually through a user or automatically. More preferably, the information of the file received automatically comes from physical sensors.

In another embodiment, the preset variables comprise: date of sampling or date of analysis, category, species and/or origin of the sample, name of the sample, microorganism and number of viable cells of the microorganism in the sample per unit area, mass, or volume.

In another embodiment, the transformation algorithm simultaneously executes one or more of the actions of:
sorting the samples into inducers, corresponding to a plant of a company, which are selected from the group consisting of: Raw Material, Process Control, and Finished Product;
completing the name of the company and plant;
completing with “Not Applicable” the species of the process control samples;
assigning a unique identifier for each analysis and each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a “less than” (<) symbol as zero, and those with a “greater than” (>) symbol as the indicated numerical value plus 1.

In another embodiment, the inducer Raw Material comprises categories that are selected from the group consisting of Animal, Vegetable, and Miscellaneous; and the inducer Finished Product comprises categories that are selected from: poultry, bovine, feline, swine, shrimp, canine, guinea pig, equine, and fish.

In another embodiment, the inducer Process Control comprises categories that are selected from the group consisting of: “hygienic zoning”, “water”, “handlers”, “environment”, “packaging material”, “product in process”, and “not applicable”. More preferably, the category “hygienic zoning” comprises the zones: “zone of surfaces in direct contact with the finished product”, “zone of surfaces close to the finished product”, “facility zone” and “non-processing zone”.

In one embodiment, the calculation algorithm includes the formula:

$$x ( a ) = \{ \begin{matrix}100 , & a \leq L \\ 0 , & a > L\end{matrix}$$

$$Safety index of a sample = \frac{{\sum }_{i = 1}^{n} {x}_{i} {w}_{i}}{{\sum }_{i = 1}^{n} {w}_{i}}$$

where L corresponds to the microbiological limit for a given microorganism, αcorresponds to the viable cell count per unit area, mass, or volume, n corresponds to the total number of samples, and w corresponds to the weight established for each microorganism.

In another embodiment, the algorithm is applied for samples corresponding to zones, categories, or inducers. More preferably, the safety indexes obtained are averaged with other calculated safety indexes corresponding to other zones, categories, inducers, or plants of a company.

Another subject matter of the present disclosure is a system for measuring and evaluating microbiological parameters and maintaining food safety in a production process of food derived from animal protein comprising:
a storage unit (1), which allows storing the verified information;
one or more processors (2), which allow the operation of the system modules and the storage unit;
a telecommunications network (3), which allow connecting the one or more processors to each other and/or to the storage unit;
a computer readable medium (4), operatively connected to the one or more processors (2) having stored instructions executable by the one or more processors (2) which, when executed, cause the one or more processors (2) to carry out a method for calculating food safety indexes, the method includes:
   a) receiving a file with tabulated information on the microbiological results of one or more stages of the production process;
   b) verifying whether the information received complies with preset variables wherein, if the information does not comply with the preset variables, it is corrected by means of a correction algorithm;
   c) transforming the information by means of a transformation algorithm;
   d) storing the information received in a data storage unit;
   e) calculating food safety indexes of the production process and/or its stages from the information received using a calculation algorithm;
   f) generating a report with the food safety indexes to determine an action in the production process and/or its stages to maintain food safety.

In one embodiment, the instructions executable by the one or more processors (2) are configured so that the one or more processors receive the file with tabulated information about preset variables, manually through a user or automatically. More preferably, the information of the file received automatically comes from physical sensors.

In another embodiment, the preset variables comprise: date of sampling or date of analysis, category, species or origin of the sample, name of the sample, microorganism and number of viable cells of the microorganism in the sample per unit area, mass, or volume.

In another embodiment, the transformation algorithm simultaneously executes one or more of the actions of:
sorting the samples into inducers, corresponding to a plant of a company, which are selected from the group consisting of: Raw Material, Process Control, and Finished Product;
completing the name of the company and plant;
completing with “Not Applicable” the species of the process control samples;
assigning a unique identifier for each analysis and each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a “less than” (<) symbol as zero, and those with a “greater than” (>) symbol as the indicated numerical value plus 1.

In another embodiment, the inducer Raw Material comprises categories that are selected from the group consisting of: Animal, Vegetable, and Miscellaneous; and the inducer Finished Product comprises categories that are selected from: poultry, bovine, feline, swine, shrimp, canine, guinea pig, equine, and fish.

The miscellaneous category corresponds to an origin that is neither animal nor vegetable, for example, chemical compounds that provide minerals such as calcium carbonate, monocalcium phosphate, bentonite, among others.

In another embodiment, the inducer Process Control comprises categories that are selected from the group consisting of: “hygienic zoning”, “water”, “handlers”, “environment”, “packaging material”, “product in process”, and “not applicable”. More preferably, the category “hygienic zoning” comprises the zones: “zone of surfaces in direct contact with the finished product”, “zone of surfaces close to the finished product”, “facility zone” and “non-processing zone”.

In one embodiment, the disclosure uses a calculation algorithm, which includes the formula:

$$x ( a ) = \{ \begin{matrix}100 , & a \leq L \\ 0 , & a > L\end{matrix}$$

$$Safety index of a sample = \frac{{\sum }_{i = 1}^{n} {x}_{i} {w}_{i}}{{\sum }_{i = 1}^{n} {w}_{i}}$$

where L corresponds to the microbiological limit for a given microorganism, a corresponds to the viable cell count per unit area, mass, or volume, n corresponds to the total number of samples, and w corresponds to the weight established for each microorganism.

In another embodiment, the algorithm is applied for samples corresponding to zones, categories, or inducers. More preferably, the safety indexes obtained are averaged with other calculated safety indexes corresponding to other zones, categories, inducers, or plants of a company.

In another embodiment, the storage unit is an external server or internal memory.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

Additional features of the disclosure will become apparent to those skilled in the art upon consideration of the following detailed description of illustrative embodiments exemplifying the best mode of carrying out the disclosure as presently perceived.

### BRIEF DESCRIPTION OF THE DRAWINGS

The foregoing and other features of the present disclosure will become more fully apparent from the following description and appended claims, taken in conjunction with the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawing, can be arranged, substituted, combined, and designed in a wide variety of different configurations, all of which are explicitly contemplated and make part of this disclosure.

The detailed description particularly refers to the accompanying figures in which:

FIG. 1 is a flow diagram showing the stages of the method of the present disclosure, in order: (a) receiving a file with tabulated information on the microbiological results of one or more stages of the production process; (b) verifying whether the information received complies with preset variables wherein, if the information does not comply with the preset variables, it is corrected by means of a correction algorithm; (c) transforming the information by means of a transformation algorithm; d) storing the information received in a data storage unit; e) calculating food safety indexes of the production process and/or its stages from the information received using a calculation algorithm; f) generating a report with the food safety indexes to define an intervention in the production process and/or its stages to maintain food safety;

FIG. 2 is a block diagram showing the different parts of the system of the present disclosure. Storage unit (1), one or more processors (2), telecommunications network (3), and computer-readable medium (4);

FIG. 3 is an organizational diagram showing the different levels of a company, specifically, plants, inducers, categories, zones, and samples. It shows the different averages that are made after the initial calculation to obtain the safety indexes for each level;

FIG. 4 is a spreadsheet showing tabulated information as received in stage (A) by the one or more processors (2);

FIG. 5 is a spreadsheet showing tabulated information after being verified, corrected, and transformed;

FIG. 6 is a spreadsheet showing tabulated information after being verified, corrected, and transformed. Microbiological limits, compliance, and average weights have been included;

FIG. 7 is a spreadsheet showing information for the inducer Process Control and the category Surface. Microbiological limits, compliance, and average weights have been included;

FIG. 8 is a bar graph showing a report of safety indexes for each sample of the category Hygienic Zoning.

FIG. 9 is a bar graph showing a report of safety indexes for each zone of the category Hygienic Zoning.

FIG. 10 is a bar graph showing a report of safety indexes for each category of the inducer Process Control, and illustrating, from left to right, safety indexes of water, handler, surface or hygienic zoning, and packaging material;

FIG. 11 is an information table showing a report of safety indexes for each sample and category of the inducer Raw Material;

FIG. 12 is an information table showing a report of safety indexes for each sample and category of the inducer Finished Product;

FIG. 13 is a bar graph showing a report of safety indexes per inducer, and illustrating, from left to right, safety indexes of process control, raw material, and finished product;

FIG. 14 is a graphical report of safety indexes per plant; and

FIG. 15 is a graphical report of a company's safety indexes.

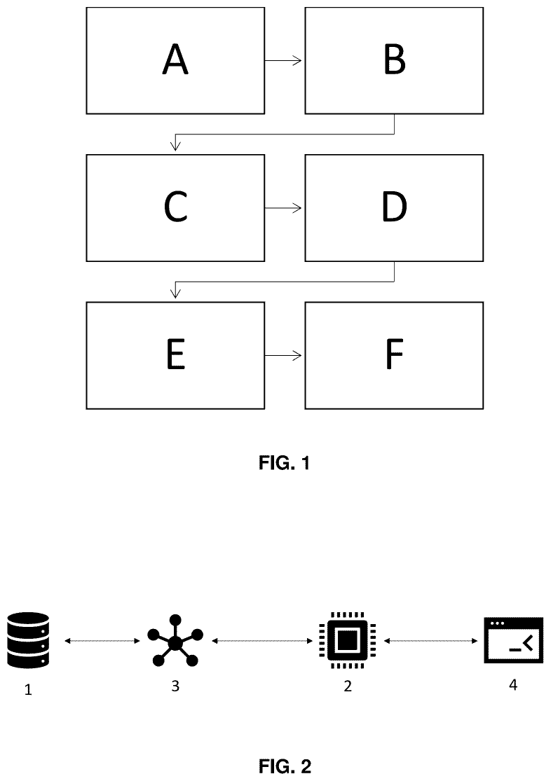

### DETAILED DESCRIPTION OF THE DISCLOSURE

The following detailed description is directed to certain specific embodiments of the technology. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the disclosure. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

The present disclosure is directed to a system and a method for the measurement, evaluation, implementation, and maintenance of the safety index for microbiological analysis for food safety for producers of food derived from animal protein. The system and method allows form the analysis of the microbiological behaviors and the actual or potential risk levels of microorganisms (bacteria and fungi) in each of the necessary production stages. For this purpose, statistical and probabilistic parameters are combined to define the different performance states in terms of quality and safety in order to achieve an optimal control of resources in the raw material, during the production process and in the finished product for making assertive and timely informed decisions based on data as evidence for the production process of food derived from animal proteins.

All technical and scientific terms used to describe the present disclosure have the same meaning understood by a person with basic knowledge in the technical field in question. However, in order to define the scope of the disclosure more clearly, a list of the terminology used in this description is included below.

An “inducer” should be understood as a stage of the production process. For each plant of a company, there can be three inducers, which include “raw material”, “process control”, and “finished product”.

“Process control categories” should be understood as all factors that directly or indirectly impact production and correspond to “hygienic zoning”, “water”, “handlers”, “environment”, “packaging material”, and “product in process”.

“Hygienic zoning” should be understood as the category corresponding to surface samples, which are classified into four zones according to their proximity to the finished product.

The “zone of surfaces in direct contact with the finished product” should be understood as the zone comprising the surface samples closest to the finished product and, therefore, representing the highest level of risk. This zone is also identified as “z1”.

The “zone of surfaces close to the finished product” should be understood as the zone comprising surface samples in non-contact, but in close proximity to the finished product. This zone is also identified as “z2”.

The “facility zone” should be understood as the zone comprising surface samples away from the finished product, such as access corridors, doors, walls, etc. This zone is also identified as “z3”.

The “non-processing zone” should be understood as the zone comprising surface samples from non-processing areas, meaning the lowest level of risk. This zone is also identified as “z4”.

“Food derived from animal protein” should be understood as any food product derived from animal protein, such as meat products, egg products, dairy products, produced by laboratory culture, among others.

“CFU” should be understood as colony forming unit, i.e., each CFU means a viable cell capable of forming a colony, and consequently the number of CFU represents the number of viable cells present in a sample. CFUs can be measured by different methods, such as plate culture, flow cytometry, among others. A viable cell count can be, for example, Enterobacteriaceae count, Escherichia coli count, yeast count, mold count, Clostridium count, Salmonella count, Mesophiles count, Coliforms count, Campylobacter count, Listeria count, Pseudomonas count, among others.

“Microbiological limit” should be understood as a limit for viable cell counts for a microorganism or group of microorganisms per unit of area, mass, or volume, which allows establishing the risk produced by a given organism in a sample, and is defined according to local and global regulations.

The “safety index of a sample” should be understood as a value calculated by a calculation algorithm that includes the formula:

$$x ( a ) = \{ \begin{matrix}100 , & a \leq L \\ 0 , & a > L\end{matrix}$$

$$Safety index of a sample = \frac{{\sum }_{i = 1}^{n} {x}_{i} {w}_{i}}{{\sum }_{i = 1}^{n} {w}_{i}}$$

where L corresponds to the microbiological limit for a given microorganism, a corresponds to the viable cell count per unit area, mass, or volume, n corresponds to the total number of samples, and w corresponds to the weight established for each microorganism.

For example, the limit L may correspond to a certain microorganism in a certain type of plant, in a certain category, in a certain hygienic zoning, for a certain species and pathogenicity.

The term “safety index” should be understood as the numerical value calculated from the formula for the safety index of a sample, or the average of several safety indexes according to a given weight. This numerical value is in a range between 0 and 100. In this context, there are safety indexes per zone, in which each safety index of a sample of the zone has the same weight in average. Then, there is a safety index per hygienic zoning category, where the weights for each safety index per zone correspond to those shown in the following Table 1.

TABLE 1

| Zone | Description | Weight |
|---|---|---|
| z1 | Surfaces in direct contact with the balanced feed | 0.4 |
| z2 | Surfaces not in contact, but very close to the product | 0.3 |
| z3 | Basic GMP, access corridors, doors, walls, etc. | 0.2 |
| z4 | Non-processing areas | 0.1 |

To calculate safety indexes for the other process control categories, i.e., water, environment, handlers, packaging material, and product in process, the safety indexes of the samples are averaged with the same weight. On the other hand, there is the safety index of the inducer Process Control, where the weights for each safety index per category correspond to those shown in the following Table 2.

TABLE 2

| Category Process Control | Weight |
|---|---|
| Handler | 0.05 |
| Packaging material | 0.05 |
| Water | 0.1 |
| Environment | 0.05 |
| Hygienic zoning | 0.5 |
| Product in process | 0.2 |

The inducers Raw Material and Finished Product are not subclassified into categories or zones; therefore, for the calculation of the safety index of each of them, the safety index of the samples corresponding to each one is averaged. On the other hand, there is the safety index per plant, where the weights for each safety index per inducer correspond to those observed in the following Table 3.

TABLE 3

| Inducer | Weight |
|---|---|
| Process control | 0.5 |
| Finished product | 0.3 |
| Raw material | 0.2 |

On the other hand, there is the safety index per company, where the weights for each safety index per plant correspond to those shown in the following Table 4.

TABLE 4

| Plant | Weight |
|---|---|
| Balanced Feed Plant | 0.1 |
| Farm | 0.05 |
| Incubator | 0.05 |
| Breeder | 0.05 |
| Processing plant | 0.15 |
| Rendering plant | 0.05 |
| Human plant | 0.2 |
| Point of sale | 0.15 |
| Laboratory | 0.05 |
| Restaurant | 0.15 |

“Preset variables” should be understood as the variables present in a file with tabulated information on the microbiological results of one or more stages of a production process. Preset variables include: date of sampling or date of analysis, category, species or origin of the sample, name of the sample, microorganism and number of viable cells of the microorganism in the sample per unit area, mass, or volume.

The term “transformation algorithm” should be understood as an algorithm that, based on tabulated information on the microbiological results of one or more stages of a production process, and that complies with the preset variables, simultaneously executes one or more of the following actions:
sorting the samples into inducers, corresponding to a plant of a company, which are selected from the group consisting of: Raw Material, Process Control, and Finished Product;
completing the name of the company and plant;
completing with “Not Applicable” the species of the process control samples;
assigning a unique identifier for each analysis and each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a “less than” (<) symbol as zero, and those with a “greater than” (>) symbol as the indicated numerical value plus 1.

The present disclosure relates to a system and a method for the measurement, implementation, and maintenance of the safety index for microbiological analysis for food safety for producers of food derived from animal protein, in order to analyze the microbiological behaviors and the actual or potential risk levels of microorganisms (bacteria and fungi) in each of the necessary production stages. For this purpose, statistical and probabilistic parameters are combined to define the different performance states in terms of quality and safety in order to achieve an optimal control of resources in the raw material, during the production process and in the finished product for making assertive and timely informed decisions based on data as evidence for the production process of food derived from animal proteins.

At the same time, the present disclosure provides precise information of the different variables that affect the stages of the production process, which allows visualizing microbiological data chronologically, per inducers, categories, samples, and types of microorganisms, and provides an orderly work plan oriented to the continuous improvement and adequate use of the resources used in the production processes for the elaboration of food derived from animal proteins in the different links of the production chain, such as the balanced feed plant, rendering plant, processing plant, egg products, meat derivatives, classification, incubation, farms, and laboratories.

The system and method of the present disclosure allow the tracing of microbiological behavior from a global-specific concept, qualifying the safety performance of a production process in wider times and, at the same time, reaching relative levels to detail the particular associated safety status. The system and method of the present disclosure are tools for optimization, control, and prevention through the real-time determination of the safety index in production processes for the production in-situ of food derived from animal protein.

The data collection is carried out in the raw material, in the processes, and in the finished product in the production of animal feed, wherein, at the same time this detailed information is processed for its respective digitalization and analysis through a mobile application that is located in a mobile or portable electronic device and a digital platform that is located on a remote server, wherein these actors (hardware, (electronic measurement and control devices in the production process), mobile application, and digital platform) are interconnected through a telecommunications network via the Internet, where the best way to analyze and digitize all the detailed information was determined, in which the sampling of the safety index for microbiological analysis is performed to make the respective reports, analysis, and statistical calculations, measurement of quantitative and qualitative microbiological parameters, results of raw material, process control (environment, water, surfaces, handlers, packaging material, and intermediate product or product in process and in test), finished product and their interactions, and at the same time visualizing all the data by means of statistical graphics in real time. This allows for observing all the processes that occur in the production areas in the plant, providing accurate information of the variables in the entire production chain, achieving a more accurate assessment of the elements found in each of the processes, thus obtaining an optimal assertive and timely decision making for the improvement of the production process in the production of animal feed, processing plant, and primary production, delivering a high quality and safe product with traceability to the final consumer.

The method of the present disclosure includes the step of receiving a file with tabulated information on the microbiological results of one or more stages of the production process, manually or automatically by physical sensors such as a flow cytometer, pH sensor, humidity sensor, water activity sensor, temperature sensor, light sensor, ATP sensor, CO₂ sensor, or flow sensor. This file can be in different formats such as Word, PDF, Excel, among others. The information contained in the file must comply with the preset variables.

Next, the information is verified by one or more processors so that it complies with the format requirements for the preset variables, and if it is verified that the information complies with these, the information is transformed by means of the transformation algorithm, and subsequently stored in a storage unit, which can be an external server, or an internal memory. In the event that the information does not comply with the format of the preset variables, it is corrected by a correction algorithm, which corrects any formatting errors or conceptual inconsistencies accordingly, prior to its transformation and storage.

Then, the sample safety indexes are calculated by one or more processors using the calculation algorithm, and the other safety indexes are calculated by one or more processors by weighting as the case may be.

The present disclosure includes a telecommunications network (3) which allows connecting the one or more processors to each other and/or to the storage unit. In this way, it is possible to carry information between these different components.

The system of the present disclosure includes mobile devices such as a cell phone, a tablet, a computer, laptop, PC, server, or other electronic device with communication elements having Internet access that are located in the same or different places. These receive, process, transform, and/or emit data to analyze and digitalize the detailed information in the place and/or location where the data collection is carried out in the respective environments or zones that are part of the production plant, providing accurate information in the data collection by measuring the safety index for the microbiological analysis obtained in each of the areas of the plant.

The system of the present disclosure includes a computer readable medium operatively connected to the one or more processors having stored instructions executable by the one or more processors which, when executed, cause the one or more processors to carry out the method of the present disclosure.

The manner in which a user may use the platform provided by the present disclosure is as follows. The client user enters the platform, this entry is made by means of identification credentials, these credentials may be a valid email and in use, the credentials were previously sent and assigned and the identification credentials were sent to the respective email of the user and the password to the user specifically. Once the user enters the platform with his/her respective username and password, the platform verifies the data entered by the user and password and determines whether or not it is a client linked to the corresponding company, then if the platform determines that the client is not linked, a message is displayed to the user indicating that the client does not exist. Subsequently, if the data corresponds to a related client, the user will gain access to the platform and a menu will be sent to the user where he/she must enter the respective information as new user or consult the information that is already available in the platform.

To enter information, the user must enter a file in the platform and can assign the following variables to the information: name of the company, name of the plant, type of plant, sample identification or sample ID, name of the sample, origin of the sample or target species of the sample, the inducer to which the sample belongs, the microorganism of the analysis, result of the analysis, and date of sampling. This information can also be entered automatically using sensors. This information is verified by the one or more processors (2), and if it is verified is sent to the storage unit (1) through the telecommunications network (3). Then, this information is processed by the one or more processors (2) using the computer readable medium (4), obtaining the results of safety indexes and safety risk.

In the following, by way of example, but not limiting the full scope of the present disclosure, the different microbiological criteria considered to define the microbiological limits used in the calculation algorithm can be observed.

### Microbiological Criteria for Balanced Feed

The microbiological criteria defined under the technical-scientific supports used as reference have application for the following species: swine, poultry, fish, canine, feline, ovine, bovine, and equine.

| Microorganism | n | m | M | c |
|---|---|---|---|---|
| Aerobic Mesophiles count cfu/g | 5 | 10,000 | 100,000 | 1 |
| Enterobacteriaceae count cfu/g | 5 | 100 | 1,000 | 1 |
| E. coli count cfu/g | 5 | <10 | N.A. | 0 |
| Mold and yeast count cfu/g | 5 | 1,000 | 10,000 | 1 |
| Sulfite-reducing Clostridium spores count cfu/g | 5 | 10 | 100 | 1 |
| Investigation of Salmonella spp in 25 g | 5 | Absence | N.A. | 0 |

where: n: number of samples; m: microbiological criterion that separates acceptable from rejectable quality; M: microbiological criterion that separates provisionally acceptable from rejectable quality; c: maximum number of rejectable sample units.

However, those used in Colombia by species according to ICA regulations are the following:

Poultry species

| Analysis | Specification | Unit |
|---|---|---|
| Mesophilic Microorganisms Count | 1,000,000 | CFU/g or ml |
| Coliform Microorganisms count | 100,000 | CFU/g or ml |
| E. coli count | 0 | CFU/g or ml |
| Sulfite-reducing Clostridium spores count | 200 | CFU/g or ml |
| Mold and yeast count | 100,000 | CFU/g or ml |
| Investigation of Salmonella spp | 0 | absence/25 g |

Swine species

| Analysis | Specification | Unit |
|---|---|---|
| Mesophilic Microorganisms Count | 10,000,000 | CFU/g or ml |
| Coliform Microorganisms count | 100,000 | CFU/g or ml |
| E. coli count | 0 | CFU/g or ml |
| Sulfite-reducing Clostridium spores count | 200 | CFU/g or ml |
| Mold and yeast count | 100,000 | CFU/g or ml |
| Investigation of Salmonella spp | 0 | absence/25 g |

Rabbit species

| Analysis | Specification | Unit |
|---|---|---|
| Mesophilic Microorganisms Count | 10,000 | CFU/g or ml |
| Coliform Microorganisms count | 500 | CFU/g or ml |
| E. coli count | 0 | CFU/g or ml |
| Sulfite-reducing Clostridium spores count | 10 | CFU/g or ml |
| Mold and yeast count | 5,000 | CFU/g or ml |
| Investigation of Salmonella spp | 0 | absence/25 g |

Canine species

| Analysis | Specification | Unit |
|---|---|---|
| Mesophilic Microorganisms Count | 50,000 | CFU/g or ml |
| Coliform Microorganisms count | 1,000 | CFU/g or ml |
| E. coli count | 0 | CFU/g or ml |
| Sulfite-reducing Clostridium spores count | 100 | CFU/g or ml |
| Mold and yeast count | 5,000 | CFU/g or ml |
| Investigation of Salmonella spp | 0 | absence/25 g |

Feline species

| Analysis | Specification | Unit |
|---|---|---|
| Mesophilic Microorganisms Count | 50,000 | CFU/g or ml |
| Coliform Microorganisms count | 1,000 | CFU/g or ml |
| E. coli count | 0 | CFU/g or ml |
| Sulfite-reducing Clostridium spores count | 100 | CFU/g or ml |
| Mold and yeast count | 5,000 | CFU/g or ml |
| Investigation of Salmonella spp | Absence | absence/25 g |

Fish species

| Analysis | Specification | Unit |
|---|---|---|
| Mesophilic Microorganisms Count | 100,000 | CFU/g or ml |
| Coliform Microorganisms count | 1,000 | CFU/g or ml |
| E. coli count | 0 | CFU/g or ml |
| Sulfite-reducing Clostridium spores count | 100 | CFU/g or ml |
| Mold and yeast count | 5,000 | CFU/g or ml |
| Investigation of Salmonella spp | Absence | absence/25 g |

The bovine, equine, and ovine species correspond to a bibliographic review for the definition of their specifications in addition to the use of the technical standard NTC 2030 for bovines. NTC 2030 and NTC 5393 for equines.

Bovine species

| Analysis | Specification | Unit |
|---|---|---|
| Mesophilic Microorganisms Count | 100,000,000 | CFU/g or ml |
| Coliform Microorganisms count | 100,000 | CFU/g or ml |
| E. coli count | 0 | CFU/g or ml |
| Sulfite-reducing Clostridium spores count | 200 | CFU/g or ml |
| Mold and yeast count | 100,000 | CFU/g or ml |
| Investigation of Salmonella spp | 0 | Absence/25 g |

Equine species

| Analysis | Specification | Unit |
|---|---|---|
| Mesophilic Microorganisms Count | 5,000,000 | CFU/g or ml |
| Coliform Microorganisms count | 10,000 | CFU/g or ml |
| E. coli count | 0 | CFU/g or ml |
| Sulfite-reducing Clostridium spores count | 100 | CFU/g or ml |
| Mold and yeast count | 10,000 | CFU/g or ml |
| Investigation of Salmonella spp | 0 | Absence/25 g |

Ovine species

| Analysis | Specification | Unit |
|---|---|---|
| Mesophilic Microorganisms Count | 1,000,000 | CFU/g or ml |
| Coliform Microorganisms count | 10,000 | CFU/g or ml |
| E. coli count | 0 | CFU/g or ml |
| Sulfite-reducing Clostridium spores count | 200 | CFU/g or ml |
| Mold and yeast count | 10,000 | CFU/g or ml |
| Investigation of Salmonella spp | 0 | Absence/25 g |

Microbiological criteria for raw material of plant origin (bibliographic review)

| Microorganism | n | m | M | c |
|---|---|---|---|---|
| Aerobic Mesophiles count cfu/g | 5 | N.A. | 100,000 | 1 |
| Enterobacteriaceae count cfu/g or Total Coliforms | 5 | N.A. | 100 | 1 |
| E. coli count cfu/g | 5 | Absence | N.A. | 0 |
| Mold and yeast count cfu/g | 5 | N.A. | 1,000 | 1 |
| Sulfite-reducing Clostridium spores count cfu/g | 5 | 10 | 100 | 1 |
| Investigation of Salmonella spp in 25 g | 5 | Absence | N.A. | 0 |

where: n: number of samples; m: microbiological criterion that separates acceptable from rejectable quality; M: microbiological criterion that separates provisionally acceptable from rejectable quality; c: maximum number of rejectable sample units.

Microbiological criteria for animal by-products
(NTC 685 and bibliographic review)

| Microorganism | n | m | M | c |
|---|---|---|---|---|
| Enterobacteriaceae count cfu/g | 5 | N.A. | 1,000 | 1 |
| E. coli count cfu/g | 5 | Absence | N.A. | 0 |
| Mold and yeast count cfu/g | 5 | N.A. | 1,000 | 1 |
| Sulfite-reducing Clostridium spores count cfu/g | 5 | N.A. | 200 | 1 |
| Investigation of Salmonella spp in 25 g | 5 | Absence | N.A. | 0 |
| Aerobic Mesophiles counts | 5 | N.A. | 100,000 | 1 |

where: n: number of samples; m: microbiological criterion that separates acceptable from rejectable quality; M: microbiological criterion that separates provisionally acceptable from rejectable quality; c: maximum number of rejectable sample units.

Miscellaneous microbiological criteria (salts,
premixtures, bibliographic review)

| Analysis | Specification | Unit |
|---|---|---|
| Mesophilic Microorganisms Count | 10,000 | cfu/g o ml |
| Coliform Microorganisms count | 10 | cfu/g o ml |
| E. coli count | 0 | cfu/g o ml |
| Sulfite-reducing Clostridium spores count | 0 | cfu/g o ml |
| Mold and yeast count | 100 | cfu/g o ml |
| Investigation of Salmonella spp | 0 | Absence/25 g |

Microbiological criteria for surface smear (applicable to
balanced feed and rendering plants under guide FSC 36 Version
7.0). Use these when the process is in stabilization.

| Microorganism | n | m | c |
|---|---|---|---|
| Enterobacteriaceae count cfu/cm² | 5 | 120 | 0 |
| E. coli count cfu/cm² | 5 | 0 | 0 |
| Sulfite-reducing Clostridium spores count cfu/cm² | 5 | 50 | 0 |
| Investigation of Salmonella spp in 20-100 cm² | 5 | Absence | 0 |

Microbiological criteria for surface smear (applicable to balanced
feed and rendering plants under guide FSC 36 Version 7.0). Use
these limits when the processes are very controlled.

| Microorganism | n | m | c |
|---|---|---|---|
| Enterobacteriaceae count cfu/cm² | 5 | 30 | 0 |
| E. coli count cfu/cm² | 5 | 0 | 0 |
| Sulfite-reducing Clostridium spores count cfu/cm² | 5 | 10 | 0 |
| Investigation of Salmonella spp in 20-100 cm2 | 5 | Absence | 0 |

where: n: number of samples; m: microbiological criterion that separates acceptable from rejectable quality; M: microbiological criterion that separates provisionally acceptable from rejectable quality; c: maximum number of rejectable sample units.

Canine and feline products

| Microorganism | m | M |
|---|---|---|
| Aerobic Mesophiles Count cfu/cm² | 500 | 5,000 |
| Enterobacteriaceae count cfu/cm² o Total Coliforms | 10 | N.A. |
| E. coli count cfu/cm² | Absence | N.A. |
| Molds and yeasts count cfu/cm² cfu/cm² | 5 | 50 |
| Sulfite-reducing Clostridium spores count cfu/cm² | 0 | N.A. |
| Investigation of Salmonella spp/cm² | Absence | N.A. |

Rabbit products

| Microorganism | m | M |
|---|---|---|
| Aerobic Mesophiles Count cfu/cm² | 10 | 100 |
| Enterobacteriaceae count cfu/cm² o Total Coliforms | 10 | N.A. |
| E. coli count cfu/cm² | Absence | N.A. |
| Molds and yeasts count cfu/cm² cfu/cm² | 5 | 50 |
| Sulfite-reducing Clostridium spores count cfu/cm² | 0 | N.A. |
| Investigation of Salmonella spp/cm² | Absence | N.A. |

Fish products

| Microorganism | m | M |
|---|---|---|
| Aerobic Mesophiles Count cfu/cm² | 100 | 1,000 |
| Enterobacteriaceae count cfu/cm² o Total Coliforms | 10 | N.A. |
| E. coli count cfu/cm² | Absence | N.A. |
| Molds and yeasts count cfu/cm² cfu/cm2 | 5 | 50 |
| Sulfite-reducing Clostridium spores count cfu/cm² | 0 | N.A. |
| Investigation of Salmonella spp/cm² | Absence | N.A. |

Bovine products

| Microorganism | m | M |
|---|---|---|
| Aerobic Mesophiles Count cfu/cm² | 10,000 | 100,000 |
| Enterobacteriaceae count cfu/cm² o Total Coliforms | 100 | 1,000 |
| E. coli count cfu/cm² | Absence | N.A. |
| Molds and yeasts count cfu/cm² cfu/cm² | 100 | 1,000 |
| Sulfite-reducing Clostridium spores count cfu/cm² | 10* | N.A. |
| Investigation of Salmonella spp/cm² | Absence | N.A. |

*Minimum 10 cfu.

Equine products

| Microorganism | m | M |
|---|---|---|
| Aerobic Mesophiles Count cfu/cm² | 5,000 | 50,000 |
| Enterobacteriaceae count cfu/cm² o Total Coliforms | 10 | 100 |
| E. coli count cfu/cm² | Absence | N.A. |
| Molds and yeasts count cfu/cm² cfu/cm² | 10 | 100 |
| Sulfite-reducing Clostridium spores count cfu/cm² | 10* | N.A. |
| Investigation of Salmonella spp/cm² | Absence | N.A. |

*Minimum 10 cfu.

Microbiological criteria for poultry processing plants

| Microorganism | n | m | M | c |
|---|---|---|---|---|
| Aerobic Mesophiles count cfu/g | 5 | 260*** | N.A. | 1 |
| Enterobacteriaceae count cfu/g | 5 | 20*** | N.A. | 1 |
| E. coli count cfu/g | 5 | <10 | N.A. | 0 |
| Investigation of Salmonella spp in 25 g | 50 | Absence | N.A. | 7 |
| Investigation for Listeria monocytogenes in 25 g | 5 | Absence* | N.A. | 0 |
| | | 100 cfu/g* | | |
| Sulfite-reducing Clostridium spores count | 5 | 10 | 100 | 1 |

where: n: number of samples; m: microbiological criterion that separates acceptable from rejectable quality; M: microbiological criterion that separates provisionally acceptable from rejectable quality; c: maximum number of rejectable sample units.
*Before the feed has left the immediate control of the company that produced it.
**Products commercialized during their shelf life.
***Results after the cooling stage.

Microbiological criteria for swine and bovine processing plants

| Microorganism | n | m | M | c |
|---|---|---|---|---|
| Aerobic Mesophiles count cfu/g | 5 | 10,000 | 100,000 | 1 |
| Enterobacteriaceae count cfu/g | 5 | 100 | 1,000 | 1 |
| E. coli count cfu/g | 5 | <10 | N.A. | 0 |
| Sulfite-reducing Clostridium spores count cfu/g | 5 | 10 | 100 | 1 |
| Investigation of Salmonella spp in 25 g | 50 | Absence | N.A. | 3 |
| Investigation for Listeria monocytogenes in 25 g | 5 | Absence | N.A. | 0 |

where: n: number of samples; m: microbiological criterion that separates acceptable from rejectable quality; M: microbiological criterion that separates provisionally acceptable from rejectable quality; c: maximum number of rejectable sample units.

Microbiological criteria for surface smear
(applicable to processing plants)

| Microorganism | m | c |
|---|---|---|
| Enterobacteriaceae count cfu/cm² | <10 | 0 |
| E. coli count cfu/cm² | 0 | 0 |
| Investigation of Listeria spp or Listeria monocytogenes | Absence | 0 |
| Investigation of Salmonella spp in 20-100 cm² | Absence | 0 |
| Sulfite-reducing Clostridium spores count cfu/cm² | <10 | 0 |
| Mesophilic count cfu/cm² | 1,000 | 1 |
| Mold and yeast count cfu/cm² | 100 | 1 |

where: m: microbiological criterion separating acceptable from rejectable quality; c: maximum number of rejectable sample units.

Microbiological criteria for water

| Microorganism | m | c |
|---|---|---|
| Aerobic Mesophiles Count cfu/mL or cm³ | 100 | 0 |
| Total Coliforms count cfu/mL or cm³ | <1 or Absence | 0 |
| E. coli count cfu/mL or cm³ | <1 or Absence | 0 |
| Investigation of Salmonella spp/mL or cm³ | Absence | 0 |
| Investigation of Pseudomonas/mL or cm | Absence | 0 |

where: m: microbiological criterion separating acceptable from rejectable quality; c: maximum number of rejectable sample units.

Microbiological criteria for handlers

| Microorganism | m | c |
|---|---|---|
| Total Coliforms count cfu/mL or cm³ | Absence* | 0 |
| E. coli count cfu/mL or cm³ | Absence | 0 |

*Depends on the type of plant where it is measured, in rendering and balanced feed plants it can be allowed up to 100 due to the handling of materials in process.
where: m: microbiological criterion that separates acceptable from rejectable quality; c: maximum number of rejectable sample units.

Microbiological criteria for farms, breeders, and hatcheries

| Microorganism | m |
|---|---|
| Identification of Clostridium perfringens | Absence |
| Investigation of Salmonella spp in 25 g | Absence |

The use of the value for “m” or for M″ will be considered from the knowledge of the process and for plants that process more than one species, the specifications should be those of the species with the highest risk.

Poultry and swine products

| Microorganism | m | M |
|---|---|---|
| Aerobic Mesophiles Count cfu/cm² | 1,000 | 10,000 |
| Enterobacteriaceae count cfu/cm² or Total coliforms | 100 | 1,000 |
| E. coli count cfu/cm² | Absence | N.A. |
| Molds and yeasts count cfu/cm² cfu/cm² | 100 | 1,000 |
| Sulfite-reducing Clostridium spores count cfu/cm² | 10* | N.A. |
| Investigation of Salmonella spp/cm² | Absence | N.A. |

*minimum 10 cfu

Microorganisms and identification

| | | | Unit of | |
|---|---|---|---|---|
| Microorganisms | Identification | Risk | Measurement | Limit |
| Enterobacteriaceae | Citrobacter spp | Low | Absence/ | Applies the one |
| | | | Presence | used for the |
| | | | | family |
| | Enterobacter spp | Low | Absence/ | Applies the one |
| | | | Presence | used for the |
| | | | | family |
| | E. coli | Medium | Absence/ | Absence |
| | | | Presence | |
| | Erwinia spp | Low | Absence/ | Applies the one |
| | | | Presence | used for the |
| | | | | family |
| | Klebsiella spp | Low | Absence/ | Applies the one |
| | | | Presence | used for the |
| | | | | family |
| | Serratia spp | Low | Absence/ | Applies the one |
| | | | Presence | used for the |
| | | | | family |
| E. coli | E. coli 0157:H7 | High | Absence/ | Absence |
| | | | Presence | |
| | E. coli STEC | High | Absence/ | Absence |
| | | | Presence | |
| Molds | Aspergillus | Medium | Absence/ | Absence |
| | | | Presence | |
| | Fusarium | Medium | Absence/ | Absence |
| | | | Presence | |
| | Penicillium | Medium | Absence/ | Absence |
| | | | Presence | |
| Listeria | Listeria | High | Absence/ | Absence |
| | monocytogenes | | Presence | |
| | Listeria ivanovii | High | Absence/ | Absence |
| | | | Presence | |
| | Listeria inocua | Medium | Absence/ | In finished |
| | | | Presence | product it is not |
| | Listeria grayi | Medium | Absence/ | pathogenic, on |
| | | | Presence | surfaces it |
| | Listeria seeligeri | Medium | Absence/ | should be |
| | | | Presence | |
| | Listeria | Medium | Absence/ | absence |
| | welshimeri | | Presence | |
| Campylobacter | Campylobacter | Medium | Absence/ | Applies the one |
| | jejuni | | Presence | used for the |
| | | | | family |
| | Campelobacter | Medium | Absence/ | Applies the one |
| | coli | | Presence | used for the |
| | | | | family |
| Pseudomonas | Pseudomonas | Medium | Absence/ | Absence |
| | aureginosa | | Presence | |
| Clostridium | Clostridium | High | Absence/ | Absence |
| | perfringens | | Presence | |
| | Clostridium | High | Absence/ | Absence |
| | botulinum | | Presence | |

Microbiological limits for zone of surfaces
in direct contact with the finished product

| Microorganism | m |
|---|---|
| Aerobic Mesophiles count cfu/20-25 cm² or 100 cm² | 1,000 |
| Enterobacteriaceae count cfu/20-25 cm² or 100 cm² o Total Coliforms | 100 |
| E. coli count cfu/20-25 cm² or 100 cm² | Absence |
| Molds and yeasts count cfu/20-25 cm² or 100 cm² | 100 |
| Sulfite-reducing Clostridium spores count cfu/20-25 cm² or 100 cm² | 10 |
| Investigation of Salmonella spp /20-25 cm² or 100 cm² | Absence |

Microbiological limits for non-contact surfaces, but very close
to the product (zone of surfaces close to the finished product)

| Microorganism | m |
|---|---|
| Aerobic Mesophiles count cfu/20-25-25-100 cm² | 10,000 |
| Enterobacteriaceae count cfu/20-25-25-100 cm² o Total coliforms | 1,000 |
| E. coli count cfu/20-25-25-100 cm² | Absence |
| Mold and yeast count cfu/20-25-100 cm² cfu/20-25-100 cm² | 1,000 |
| Sulfite-reducing Clostridium spores count cfu/20-25-25-100 cm² | 10 |
| Investigation of Salmonella spp/20-25-100 cm² | Absence |

Microbiological limits for facility areas, access corridors, doors, walls, intermediate
process areas prior to application of microbiological control treatments

| Microorganism | m |
|---|---|
| Aerobic Mesophiles count cfu/20-25-25-100 cm² | 100,000 |
| Enterobacteriaceae count cfu/20-25-25-100 cm² o Total coliforms | 10,000 |
| E. coli count cfu/20-25-25-100 cm2 | Absence |
| Mold and yeast count cfu/20-25-100 cm² cfu/20-25-100 cm² | 10,000 |
| Sulfite-reducing Clostridium spores count cfu/20-25-25-100 cm² | 100 |
| Investigation of Salmonella spp/20-25-100 cm² | Absence |

Microbiological limits for zone of non-processing areas

| Microorganism | m |
|---|---|
| Aerobic Mesophiles count cfu/20-25-25-100 cm² | 1,000,000 |
| Enterobacteriaceae count cfu/20-25-25-100 cm² o Total coliforms | 10,000 |
| E. coli count cfu/20-25-25-100 cm² | Absence |
| Mold and yeast count cfu/20-25-100 cm² cfu/20-25-100 cm² | 10,000 |
| Sulfite-reducing Clostridium spores count cfu/20-25-100 cm² | 100 |
| Investigation of Salmonella spp/20-25-100 cm² | Absence |

Microbiological limits for water

| Microorganism | m | c |
|---|---|---|
| Aerobic Mesophiles Count cfu/mL or cm³ | 100 | 0 |
| Total Coliforms count cfu/mL or cm³ | <1 or Absence | 0 |
| E. coli count cfu/mL or cm³ | <1 or Absence | 0 |
| Investigation of Salmonella spp/mL or cm³ | Absence | 0 |
| Investigation of Pseudomonas/ mL or cm³ | Absence | 0 |

*The reference used for these parameters is Resolution 2115:2007, a standard for drinking water for human consumption used at industrial level for both animals and humans for the microbiological qualification of water.

Microbiological limits for handlers

| Microorganism | m | c |
|---|---|---|
| Total Coliforms count cfu/mL or cm³ | 100 | 0 |
| E. coli count cfu/mL or cm³ | Absence | 0 |

Microbiological limits for packaging material

| Microorganism | m | c |
|---|---|---|
| Aerobic Mesophiles Count cfu/mL or cm³ | <100 | 0 |
| Total Coliforms count cfu/mL or cm³ | Absence | 0 |
| E. coli count cfu/mL or cm³ | Absence | 0 |
| Investigation of Salmonella spp/mL or cm³ | Absence | 0 |
| Mold and yeast count/mL or cm³ | <100 | 0 |
| Sulfite-reducing Clostridium spores count/mL or cm³ | Absence | 0 |

### Microbiological Limits for Environment

It depends on the process control, therefore, in the calculation it should be qualified under the condition of >0 and include ranges to measure compliance since there is no reference for the qualification and it is directly related to the controls of each particular process.

Now, according to national standard UNE 100012:2005 (Cleaning of Ventilation and Air Conditioning Systems), which aims to assess ventilation and air conditioning systems, it is indicated that the total mesophilic microbiota should be less than 800 cfu/m³. After the cleaning process, the value should be 100 cfu/m³ and <100 cfu/m³ after disinfection processes. Values of 100 will be used in balanced feed plants, point of sale, processing plant, and meat derivatives plants, of 1000 cfu/m³ in rendering plants and farms. <100 cfu/m³ in hatchery indicates presence of pathogens.

### Ranges for Intervention According to the Safety Index

Additionally, interpretations of the safety indexes obtained are established, so that lower safety index values represent a higher microbiological risk. These interpretations are defined by the following ranges, which determine suggested intervention levels.

| Score | Intervention |
|---|---|
| 0-69 | Total |
| 70-85 | Process |
| 86-95 | Local |
| >95 | None |

| Concern derived | The conditions | The conditions | The conditions |
|---|---|---|---|
| from health | reduce the level of | not change the | increase the level |
| hazard and use | concern* | level of concern | of concern |
| Utility | Increases shelf life | No changes | Reduces shelf life |
| | Case 1 | Case 2 | Case 3 |
| | three classes | three classes | three classes |
| | n: 5, c: 3 | n: 5, c: 2 | n: 5, c: 1 |
| Indicators | Reduces hazard | No changes | Increases hazard |
| | Case 4 | Case 5 | Case 6 |
| | three classes | three classes | three classes |
| | n: 5, c: 3 | n: 5, c: 2 | n: 5, c: 1 |
| Moderate Hazard | Case 7 | Case 8 | Case 9 |
| | three classes | three classes | three classes |
| | n: 5, c: 2 | n: 5, c: 1 | n: 10, c: 1 |
| Serious hazard | Case 10 | Case 11 | Case 12 |
| | two classes | two classes | two classes |
| | n: 5, c: 0 | n: 5, c: 0 | n: 20, c: 0 |
| Critical hazard | Case 13 | Case 14 | Case 15 |
| | two classes | two classes | two classes |
| | n: 15, c: 0 | n: 30, c: 0 | n: 60, c: 0 |

*Normal expected food handling and consumption conditions after sampling.
The most rigorous sampling plans will be applied to the most sensitive foods intended for susceptible populations.

Example based on compliance with the type of sampling to be used. It is clarified that this is a proposal; according to the plant condition it should be configured what type of sampling should be executed depending on the established percentage ranges, this table corresponds to a guide.

| % of compliance | Type of sampling |
|---|---|
| 0-50 | Case 15 |
| 51-70 | Case 14 |
| 71-90 | Case 13 |
| >90 | Case 11 |

| | | | | | General inspection | | |
|---|---|---|---|---|---|---|---|
| | Special inspection levels | | | | levels | | |
| Batch size | S-1 | S-2 | S-3 | S-4 | I | II | III |
| 2 to 8 | A | A | A | A | A | A | B |
| 9 to 15 | A | A | A | A | A | B | C |
| 16 to 25 | A | A | B | B | B | C | D |
| 26 to 50 | A | B | B | C | C | D | E |
| 51 to 90 | B | B | C | C | C | E | F |
| 91 to 150 | B | B | C | D | D | F | G |
| 151 to 280 | B | C | D | E | E | G | H |
| 281 to 500 | B | C | D | E | F | H | J |
| 501 to 1,200 | C | C | E | F | G | J | K |
| 1,201 to 3,200 | C | D | E | G | H | K | L |
| 3,201 to 10,000 | C | D | F | G | J | L | M |
| 10,001 to 35,000 | C | D | F | H | K | M | N |
| 35,001 to 150,000 | D | E | G | J | L | N | P |
| 150,001 to 500,000 | D | E | G | J | M | P | Q |
| 500,001 or more | D | E | H | K | N | Q | R |

Using the results provided, the present disclosure generates a report with the food safety risk to define an intervention based on “Guidance for Developing, Documenting, Implementing and Maintaining the Quality and Food Safety Program. FCS36 V7, Safety feed and Safety Food”, to the production process and/or its stages to maintain food safety.

The following examples are intended to illustrate the disclosure and embodiments, but under no circumstances should they be considered to restrict the scope of the disclosure, which will be defined by the wording of the claims appended hereto.

### EXAMPLES

### Example 1. Calculation of the Safety Index of a Company

In order to maintain the food safety of the products of a company producing food derived from animal protein, the technology of the present disclosure was implemented to determine the safety index of the company. The company consists of a balanced feed plant, a farm, and a processing plant.

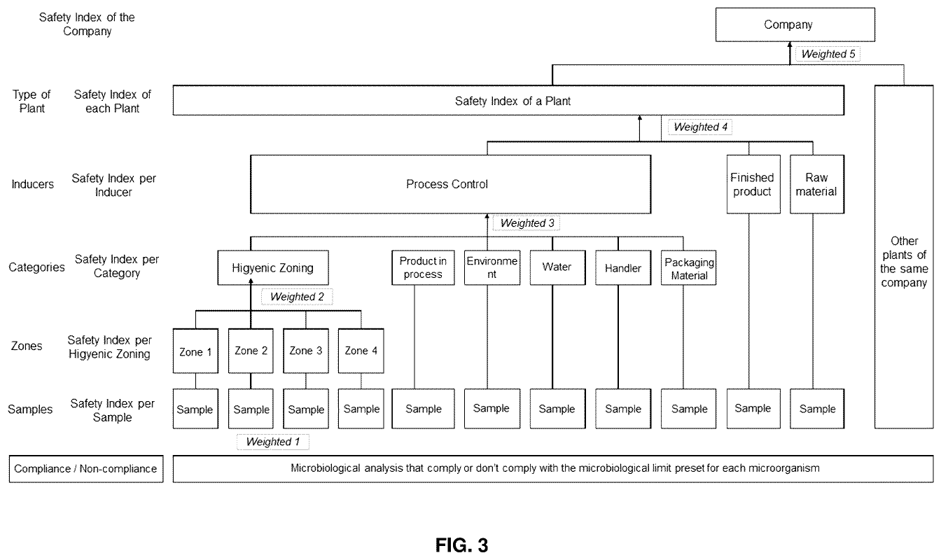

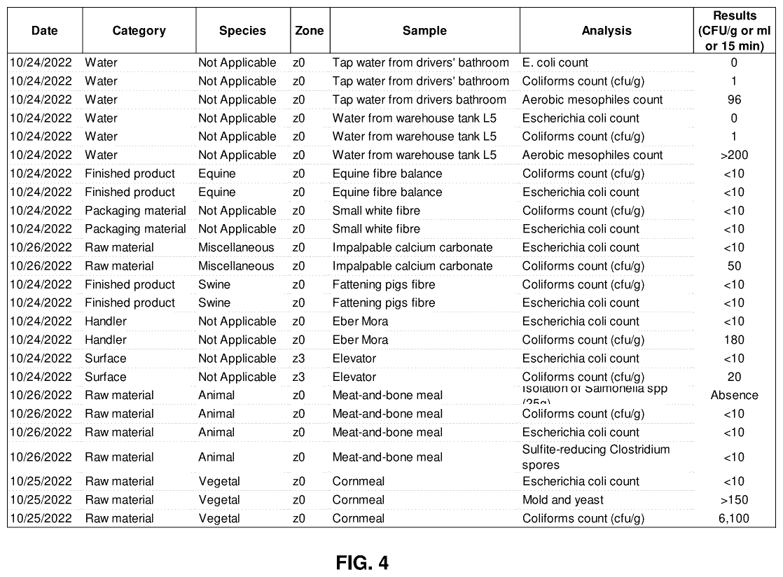

Company A took 18 samples from its balanced feed plant during its production process in October 2022 from various inducers from the plant including process control 10, finished product 12, and raw material 14 and from several categories including water 16, packaging material 18, and handler 20, as shown in FIG. 3. The samples were taken to a microbiology laboratory where they were analyzed to detect, identify, and/or quantify microorganisms of interest in the samples. A total of 44 analyses were performed. The results were tabulated and saved in an Excel file, which can be seen in FIG. 4 and includes the date, category, species, zone, where the sample was taken, the analysis performed, and the results.

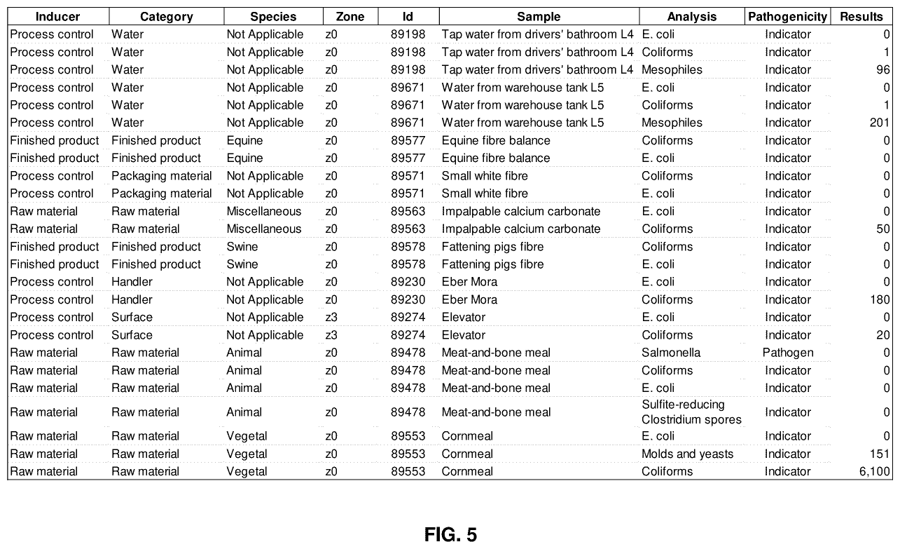

Then, the file with tabulated information on the microbiological results was verified, corrected, transformed, and stored in a server by a processor. The corrected and transformed Excel file can be seen in FIG. 5, which includes an updated zone, ID number, and pathogenicity and result determined by use of.

Then, the calculation algorithm was used, where it was first determined that when the result (number of viable cells of a microorganism in a sample) is less than or equal to the preset limit, it is complying, otherwise, it is not complying.

$$x ( a ) = \{ \begin{matrix}100 , & a \leq L \\ 0 , & a > L\end{matrix}$$

where α corresponds to the viable cell count per unit area, mass, or volume,

Then, the following algorithm is applied:

$$Safety index of a sample = \frac{{\sum }_{i = 1}^{n} {x}_{i} {w}_{i}}{{\sum }_{i = 1}^{n} {w}_{i}}$$

wherein L corresponds to the microbiological limit for a given microorganism, n corresponds to the total number of samples, and w corresponds to the weight established for each microorganism.

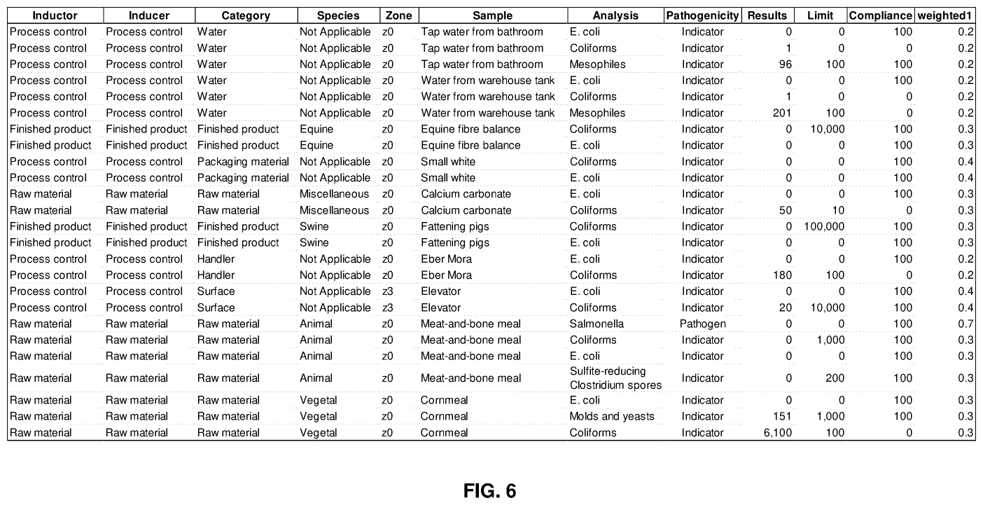

The algorithm is applied according to the weights established for each microorganism, and the safety index of the sample is obtained. FIG. 6 shows the results of the calculation of compliance or non-compliance and the weights for each analysis. FIG. 6 includes columns for the results of the algorithm including the microbiological limit, compliance and how it is weighted.

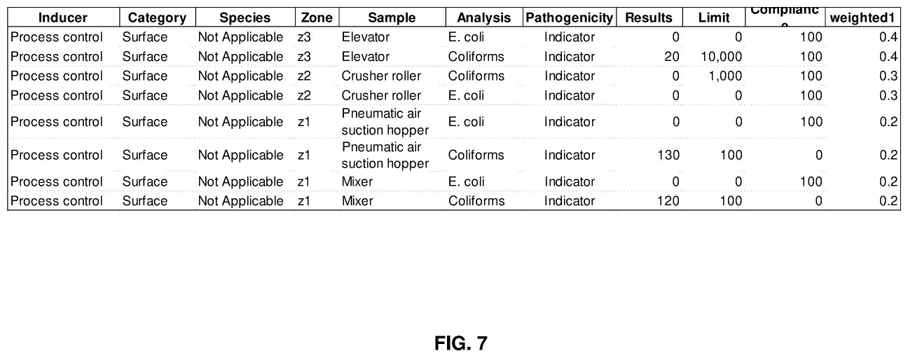

Then, the safety index was calculated for the different levels, starting with the safety index for hygienic zoning. FIG. 7 shows the results of the calculation of compliance or non-compliance and the weights for each analysis of the hygienic zoning, which were used to calculate the safety indexes for each zone according to the following calculation to obtain the safety index for the hygienic zoning.

$$Safety index ELEVATOR = \frac{\frac{100 * 0.4}{2} + \frac{100 * 0.4}{2}}{\frac{0.4}{2} + \frac{0.4}{2}} = 100.0$$

$$Safety index CRUSHER ROLLER = \frac{\frac{100 * 0.3}{2} + \frac{100 * 0.3}{2}}{\frac{0.3}{2} + \frac{0.3}{2}} = 100.0$$

$$Safety index HOPPER = \frac{\frac{100 * 0.2}{2} + \frac{0 * 0.2}{2}}{\frac{0.2}{2} + \frac{0.2}{2}} = 50.0$$

$$Safety index MIXER = \frac{\frac{100 * 0.2}{2} + \frac{0 * 0.2}{2}}{\frac{0.2}{2} + \frac{0.2}{2}} = 50.0$$

$$Safety index zone 1 = \frac{50 + 50}{2} = 50.0$$

$$Safety index zone 2 = \frac{100}{1} = 100.0$$

$$Safety index zone 3 = \frac{100}{1} = 100.0$$

$$Safety index hygienic zoning = \frac{50 * 0.4 + 100 * 0.3 + 100 * 0.2}{0.4 + 0.3 + 0.2} = 77.8$$

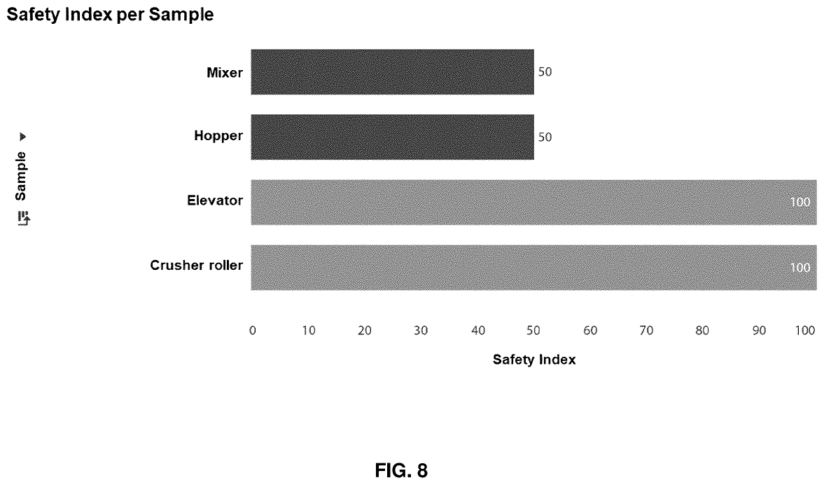

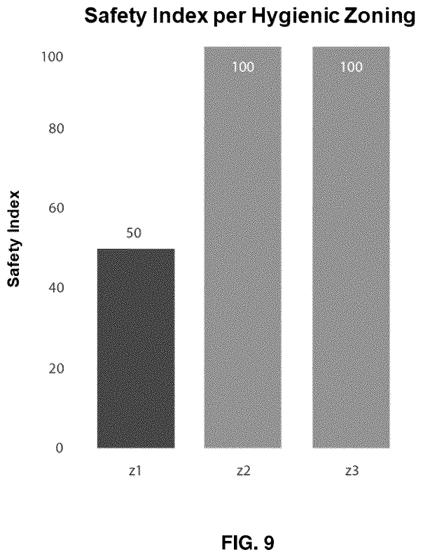

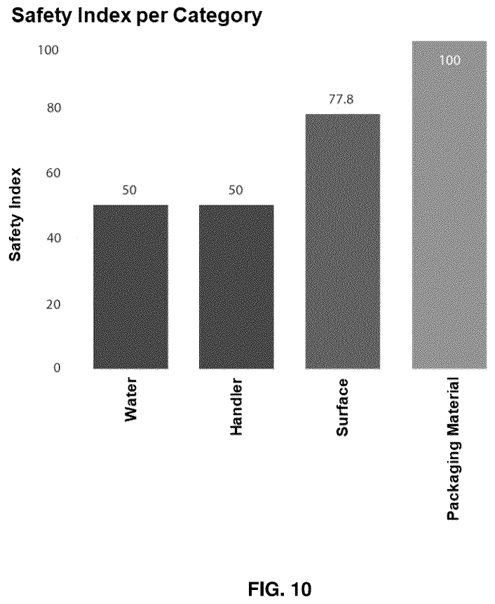

FIG. 8 illustrates a bar graph that shows the safety indexes for each sample and illustrates the safety index for samples from a mixer, hopper, elevator and crusher roller that are part of the hygienic zoning. The mixer, hopper, elevator and crusher roller samples are classified under four different hygienic zones. The surfaces in the present example are classified into z1, z1, z3 and z2, respectively FIG. 9 illustrates a bar graph that shows the safety indexes for each hygienic zone of FIG. 3. The Safety Index is a weighted average that indicates the microbiological proficiency of a plant. Each bar of the graph represents the safety index for the samples that are included in each hygienic zone. Then, the safety index of the other process control categories, such as water, handler and packing material was calculated by averaging the indexes corresponding to the samples of each one of them. FIG. 10 shows the safety indexes, for each category. Next, the safety index for the inducer Process Control was calculated by weighting the safety indexes of the different categories (water, handler, surface, packaging material) according to the following calculation.

$$Process control index = \frac{50 * 0.1 + 50 * 0.05 + 77.8 * 0.5 + 100 * 0.05}{0.1 + 0.05 + 0.5 + 0.05} = 73.4$$

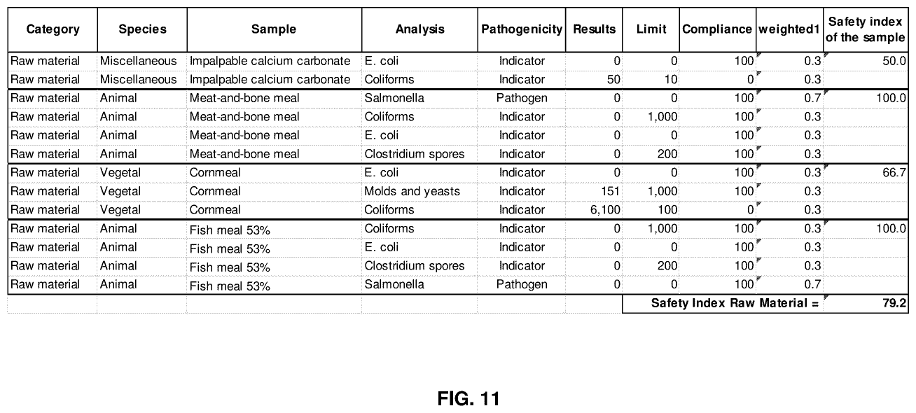

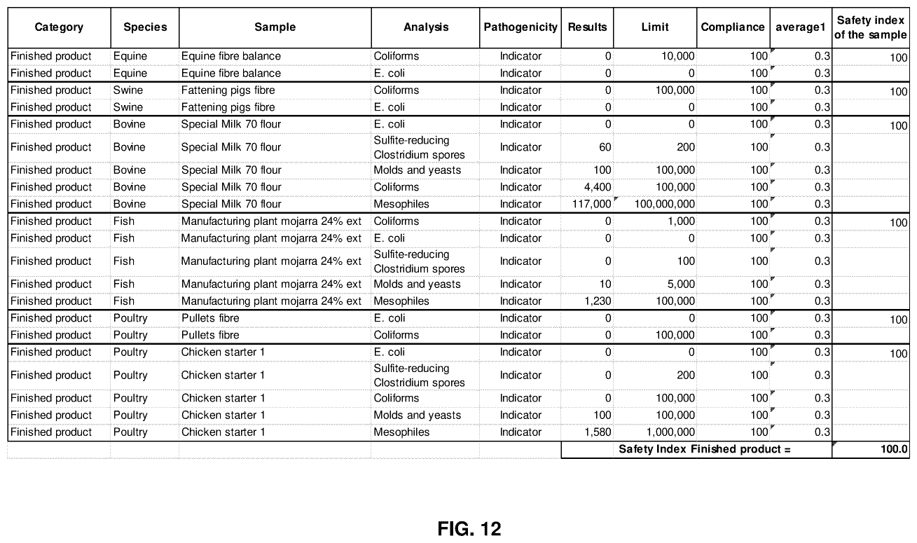

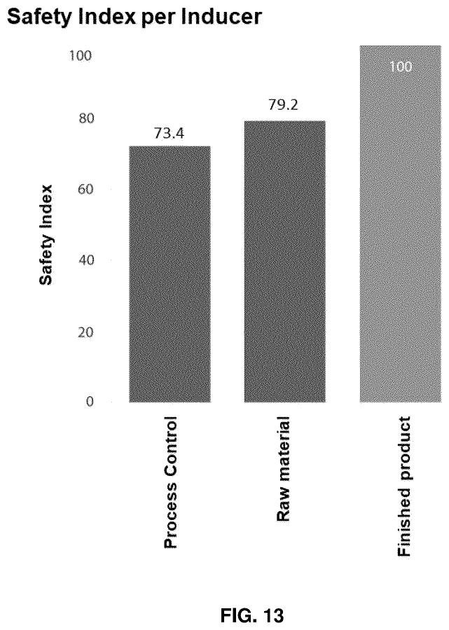

The safety index for the inducers raw material and finished product of FIG. 13 were then calculated by averaging the safety index of the corresponding samples as shown below. The results of the calculation for raw material can be seen in FIG. 11 and for finished product in FIG. 12.

$$Safety index raw material = \frac{50 + 100 + 66.7 + 100}{4} = 79.2$$

$$Safety index finished product = \frac{100 + 100 + 100 + 100 + 100 + 100}{6} = 100$$

Finally, the safety index of each inducer was obtained, which can be seen in FIG. 13.

Using these results, according to the established weights, the safety index of the plant was calculated according to the following calculation.

$$Safety index plant = \frac{73.4 * 0.5 + 79.2 * 0.2 + 100 * 0.3}{0.5 + 0.2 + 0.3} = 82.5$$

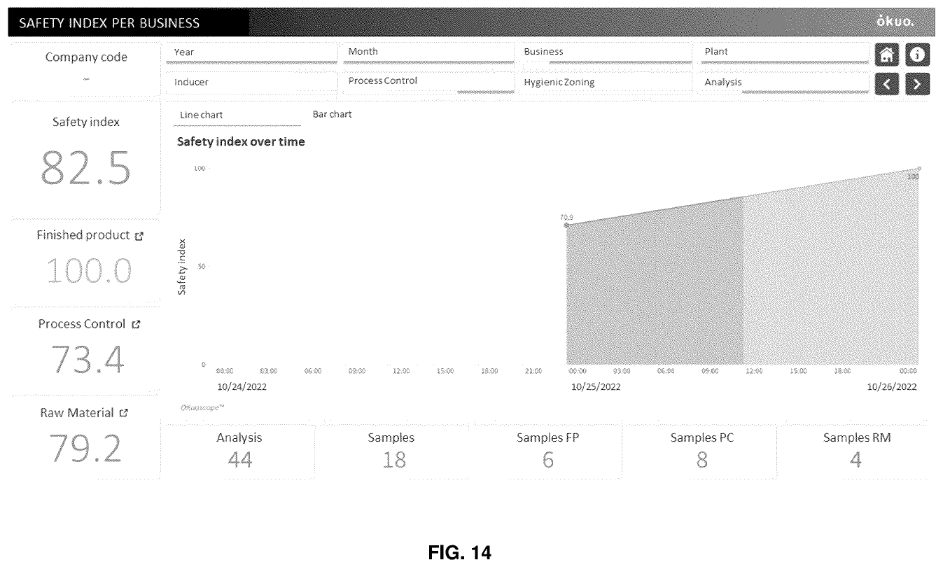

From this result, a report was generated in real time to show the safety index for each plant on a day to day and hourly basis. Real time means that as soon as new information is uploaded the safety index is recalculated and the new value is shown immediately. This report can be seen in FIG. 14. The microbiological results are taken from a laboratory report. The process can take 3-5 days after the samples arrive in the lab.

Using these results, the safety index of the company was calculated according to the established weights, as can be seen in the following calculation.

$$Safety index Company A = \frac{87 * 0.05 + 40 * 0.15 + 82.5 * 0.1}{0.05 + 0.15 + 0.3} = 62.0$$

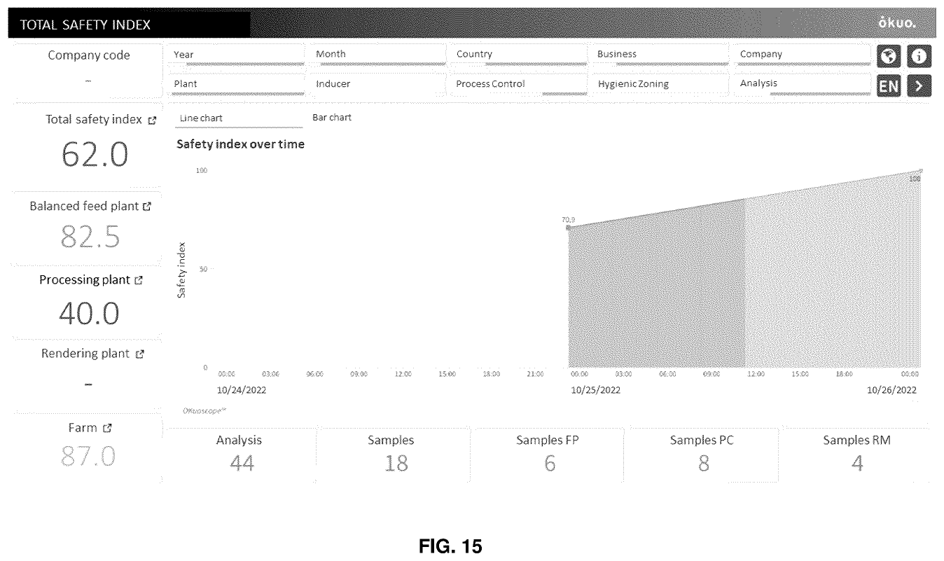

A report was generated from these results, which can be seen in FIG. 15.

Various features of the disclosure have been particularly shown and described in connection with the illustrative embodiment of the disclosure, however, it must be understood that these particular arrangements may merely illustrate, and that the disclosure is to be given its fullest interpretation within the terms of the appended claims.

It will be appreciated by those skilled in the art that various modifications and changes may be made without departing from the scope of the described technology. Such modifications and changes are intended to fall within the scope of the embodiments. It will also be appreciated by those skilled in the art that parts included in one embodiment are interchangeable with other embodiments; one or more parts from a depicted embodiment may be included with other depicted embodiments in any combination. For example, any of the various components described herein and/or depicted in the figures may be combined, interchanged or excluded from other embodiments.

Any processes or steps of any flow charts described and/or shown herein are illustrative only. A person of skill in the art will understand that the steps, decisions, and processes embodied in the flowcharts described herein may be performed in an order other than that described herein. Thus, the particular flowcharts and descriptions are not intended to limit the associated processes to being performed in the specific order described.

With respect to the use of substantially any plural and/or singular terms herein, those having skill in the art may translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific embodiments disclosed herein, but that it cover all modifications and alternatives coming within the true scope and spirit of the disclosure as embodied in the attached claims.

Example 1 includes a method for measuring and evaluating microbiological parameters and maintaining food safety in a production process for food derived from animal protein, comprising the steps of:
a) receiving a file with tabulated information on the microbiological results of one or more stages of the production process;
b) verifying whether the information received complies with preset variables wherein, if the information does not comply with the preset variables, it is corrected by means of a correction algorithm;
c) transforming the information by means of a transformation algorithm;
d) storing the information received in a data storage unit;
e) calculating food safety indexes of the production process from the information received using a calculation algorithm;
f) generating a report with the food safety indexes to determine an action in the production process to maintain food safety.

Example 2 includes the subject matter of Example 1, and wherein the file is received manually through a user or automatically.

Example 3 includes the subject matter of claim 2, and wherein the information from the file received is automatically provided by physical sensors.

Example 4 includes the subject matter of Example 1, and wherein the preset variables include date of sampling or date of analysis, category, and species.

Example 5 includes the subject matter of Example 1, and wherein the preset variables include origin of the sample, name of the sample, microorganism, and number of viable cells of the microorganism in the sample per unit area, mass, or volume.

Example 6 includes the subject matter of Example 1, and wherein the transformation algorithm simultaneously executes at least one of the following actions:
sorting the samples into inducers, corresponding to a plant of a company, which are selected from the group consisting of: Raw Material, Process Control, and Finished Product;
completing the name of the company and plant;
completing with “Not Applicable” the species of the process control samples;
assigning a unique identifier for each analysis and each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a less than (<) symbol as zero and those with a greater than (>) symbol as the indicated numerical value plus 1.

Example 7 includes the subject matter of Example 6, and wherein the inducer raw material comprises categories that are selected from the group consisting of animal, vegetable, and miscellaneous; and the inducer finished product comprises categories that are selected from the group consisting of poultry, bovine, feline, swine, shrimp, canine, guinea pig, equine, and fish.

Example 8 includes the subject matter of Example 6, and wherein the inducer process control comprises categories that are selected from the group consisting of hygienic zoning, water, handlers, environment, packaging material, product in process, and not applicable.

Example 9 includes the subject matter of Example 8, and wherein the category hygienic zoning comprises the zones: zone of surfaces in direct contact with the finished product, zone of surfaces close to the finished product, facility zone and non-processing zone.

Example 10 includes the subject matter of Example 1, and wherein the calculation algorithm includes the formula:

$$x ( a ) = \{ \begin{matrix}100 , & a \leq L \\ 0 , & a > L\end{matrix}$$

$$Safety index of a sample = \frac{{\sum }_{i = 1}^{n} {x}_{i} {w}_{i}}{{\sum }_{i = 1}^{n} {w}_{i}}$$

where L corresponds to the microbiological limit for a given microorganism, a corresponds to the viable cell count per unit area, mass, or volume, n corresponds to the total number of samples, and w corresponds to the weight established for each microorganism.

Example 11 includes the subject matter of Example 10, and wherein the algorithm is applied for samples corresponding to zones, categories, or inducers.

Example 12 includes the subject matter of Example 11, and wherein the safety indexes obtained are averaged with other calculated safety indexes corresponding to other zones, categories, inducers, or plants of a company.

Example 13 includes a system for measuring and evaluating microbiological parameters and maintaining food safety in a production process of food derived from animal protein, comprising:
a storage unit, which allows storing verified information;
one or more processors, which allow the operation of system modules and the storage unit;
a telecommunications network which allows connecting the one or more processors to each other and/or to the storage unit;
a computer readable medium, operatively connected to the one or more processors having stored instructions executable by the one or more processors which, when executed, cause the one or more processors to carry out a method for calculating food safety indexes, the method including:
   a) receiving a file with tabulated information on the microbiological results of one or more stages of the production process;
   b) verifying whether the information received complies with preset variables wherein, if the information does not comply with the preset variables, it is corrected by means of a correction algorithm;
   c) transforming the information by means of a transformation algorithm;
   d) storing the information received in a data storage unit;
   e) calculating food safety indexes of the production process from the information received using a calculation algorithm;
   f) generating a report with the food safety indexes to determine an action in the production process and/or its stages to maintain food safety.

Example 14 includes the subject matter of Example 13, and wherein the instructions executable by the one or more processors (2) are configured so that the one or more processors receive the file with tabulated information about preset variables, manually through a user or automatically.

Example 15 includes the subject matter of Example 14, and wherein the information of the file received is automatically provided by physical sensors.

Example 16 includes the subject matter of Example 13, and wherein the preset variables include: date of sampling or date of analysis, category, species or origin of the sample, name of the sample, microorganism and number of viable cells of the microorganism in the sample per unit area, mass, or volume.

Example 17 includes the subject matter of Example 13, and wherein the transformation algorithm simultaneously executes one or more of the actions of:
sorting the samples into inducers, corresponding to a plant of a company, which are selected from the group consisting of: raw material, process control, and finished product;
completing the name of the company and plant;
completing with not applicable the species of the process control samples;
assigning a unique identifier for analysis and for each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a “less than” (<) symbol as zero, and those with a “greater than” (>) symbol as the indicated numerical value plus 1.

Example 18 includes the subject matter of Example 17, and wherein the inducer raw material comprises categories that are selected from the group consisting of animal, vegetable, and miscellaneous; and the inducer Finished Product comprises categories that are selected from the group consisting of poultry, bovine, feline, swine, and shrimp.

Example 19 includes the subject matter of Example 17, and wherein the inducer process control comprises categories that are selected from the group consisting of hygienic zoning, water, handlers, environment, packaging material, product in process, and not applicable.

Example 20 includes the subject matter of Example 18, and wherein the category hygienic zoning comprises the zones: zone of surfaces in direct contact with the finished product, zone of surfaces close to the finished product, facility zone and non-processing zone.

Example 21 includes the subject matter of Example 13, and wherein the calculation algorithm includes the formula:

$$x ( a ) = \{ \begin{matrix}100 , & a \leq L \\ 0 , & a > L\end{matrix}$$

$$Safety index of a sample = \frac{{\sum }_{i = 1}^{n} {x}_{i} {w}_{i}}{{\sum }_{i = 1}^{n} {w}_{i}}$$

where L corresponds to the microbiological limit for a given microorganism, a corresponds to the viable cell count per unit area, mass, or volume, n corresponds to the total number of samples, and w corresponds to the weight established for each microorganism.

Example 22 includes the subject matter of Example 21, and wherein the algorithm is applied for samples corresponding to zones, categories, or inducers.

Example 23 includes the subject matter of Example 22, and wherein the safety indexes obtained are averaged with other calculated safety indexes corresponding to other zones, categories, inducers, or plants of a company.

Example 24 includes the subject matter of Example 13, wherein the storage unit is an external server or internal memory.

What is claimed is:

1. A method for measuring and evaluating microbiological parameters and maintaining food safety in a production process for food derived from animal protein, comprising the steps of:
a) receiving a file with tabulated information on microbiological results of one or more stages of the production process;
b) verifying whether the tabulated information received complies with preset variables wherein, if the information does not comply with the preset variables, the information is corrected by means of a correction algorithm to form corrected information;
c) transforming the corrected information by means of a transformation algorithm to form completed information;
d) storing the completed information received in a data storage unit;
e) calculating food safety indexes of the production process from the completed information received using a calculation algorithm;
f) generating a report with the food safety indexes to determine an action in the production process to maintain food safety.

2. The method according to claim 1, wherein the file is received manually through a user or automatically.

3. The method according to claim 2, wherein the information from the file received is automatically provided by physical sensors.

4. The method according to claim 1, wherein the preset variables include date of sampling or date of analysis, category, and species.

5. The method according to claim 1, wherein the preset variables include origin of the sample, name of the sample, microorganism, and number of viable cells of the microorganism in the sample per unit area, mass, or volume.

6. The method according to claim 1, wherein the transformation algorithm simultaneously executes at least one of the following actions:
sorting the samples into inducers, corresponding to a plant of a company, which are selected from the group consisting of: raw material, process control, and finished product;
completing the name of the company and plant;
completing with “not applicable” the species of the process control samples;
assigning a unique identifier for each analysis and each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a less than (<) symbol as zero and those with a greater than (>) symbol as the indicated numerical value plus 1.

7. The method according to claim 6, wherein the inducer raw material includes categories that are selected from the group consisting essentially of animal, vegetable, and miscellaneous; and the inducer finished product comprises categories that are selected from the group consisting essentially of poultry, bovine, feline, swine, shrimp, canine, guinea pig, equine, and fish.

8. The method according to claim 6, wherein the inducer process control includes categories that are selected from the group consisting essentially of hygienic zoning, water, handlers, environment, packaging material, and product in process.

9. The method according to claim 8, wherein the category hygienic zoning includes the zones: zone of surfaces in direct contact with the finished product, zone of surfaces close to the finished product, facility zone and non-processing zone.

10. The method according to claim 1, wherein the calculation algorithm includes the formula:
$$x ( a ) = \{ \begin{matrix}100 , & a \leq L \\ 0 , & a > L\end{matrix}$$
$$Safety index of a sample = \frac{{\sum }_{i = 1}^{n} {x}_{i} {w}_{i}}{{\sum }_{i = 1}^{n} {w}_{i}}$$
where L corresponds to the microbiological limit for a given microorganism, a corresponds to the viable cell count per unit area, mass, or volume, n corresponds to the total number of samples, and w corresponds to the weight established for each microorganism.

11. The method according to claim 10, wherein the algorithm is applied for samples corresponding to zones, categories, or inducers.

12. The method according to claim 11, wherein the safety indexes obtained are averaged with other calculated safety indexes corresponding to other zones, categories, inducers, or plants of a company.

13. A system for measuring and evaluating microbiological parameters and maintaining food safety in a production process of food derived from animal protein, comprising:
a storage unit, which allows storing verified information;
one or more processors, which allow the operation of system modules and the storage unit;
a telecommunications network which allows connecting the one or more processors to each other and/or to the storage unit;
a computer readable medium, operatively connected to the one or more processors having stored instructions executable by the one or more processors which, when executed, cause the one or more processors to carry out a method for calculating food safety indexes, the method including:
a) receiving a file with tabulated information on the microbiological results of one or more stages of the production process;
b) verifying whether the information received complies with preset variables wherein, if the information does not comply with the preset variables, it is corrected by means of a correction algorithm to form corrected information;
c) transforming the corrected information by means of a transformation algorithm to form completed information;
d) storing the completed information received in a data storage unit;
e) calculating food safety indexes of the production process from the completed information received using a calculation algorithm;
f) generating a report with the food safety indexes to determine an action in the production process and/or its stages to maintain food safety.

14. The system according to claim 13, wherein the instructions executable by the one or more processors are configured so that the one or more processors receive the file with tabulated information about preset variables, manually through a user or automatically.

15. The system according to claim 14, wherein the information of the file received is automatically provided by physical sensors.

16. The system according to claim 13, wherein the preset variables include: date of sampling or date of analysis, category, species or origin of the sample, name of the sample, microorganism and number of viable cells of the microorganism in the sample per unit area, mass, or volume.

17. The system according to claim 13, wherein the transformation algorithm simultaneously executes one or more of the following actions:
sorting the samples into inducers, corresponding to a plant of a company, which are selected from the group consisting of: raw material, process control, and finished product;
completing the name of the company and plant;
completing with not applicable the species of the process control samples;
assigning a unique identifier for analysis and for each sample;
standardizing the names of the microorganisms;
identifying whether each analysis performed corresponds to a pathogenic microorganism or indicator; and
capturing the number of viable cells of the microorganisms in the sample per unit area, mass, or volume, considering those results that include a “less than” (<) symbol as zero, and those with a “greater than” (>) symbol as the indicated numerical value plus 1.

18. The system according to claim 17, wherein the inducer raw material comprises categories that are selected from the group consisting essentially of animal, vegetable, and miscellaneous; and the inducer Finished Product comprises categories that are selected from the group consisting essentially of poultry, bovine, feline, swine, and shrimp.

19. The system according to claim 17, wherein the inducer process control comprises categories that are selected from the group consisting essentially of hygienic zoning, water, handlers, environment, packaging material, and product in process.

20. The system according to claim 18, wherein the category hygienic zoning comprises the zones: zone of surfaces in direct contact with the finished product, zone of surfaces close to the finished product, facility zone and non-processing zone.

21. The system according to claim 13, wherein the calculation algorithm includes the formula:
$$x ( a ) = \{ \begin{matrix}100 , & a \leq L \\ 0 , & a > L\end{matrix}$$
$$Safety index of a sample = \frac{{\sum }_{i = 1}^{n} {x}_{i} {w}_{i}}{{\sum }_{i = 1}^{n} {w}_{i}}$$
where L corresponds to the microbiological limit for a given microorganism, a corresponds to the viable cell count per unit area, mass, or volume, n corresponds to the total number of samples, and w corresponds to the weight established for each microorganism.

22. The system according to claim 21, wherein the algorithm is applied for samples corresponding to zones, categories, or inducers.

23. The system according to claim 22, wherein the safety indexes obtained are averaged with other calculated safety indexes corresponding to other zones, categories, inducers, or plants of a company.

24. The system according to claim 13, wherein the storage unit is an external server or internal memory.
